(19) United States
(12) Patent Application Publication
(10) Pub. No.: US 2003/0236511 A1
(43) Pub. Date: Dec. 25, 2003

(54) Compressed absorbent web

(76) Inventors: Archie L. Jones, Somerset, NJ (US); Lai-Hing Louie, Kendall Park, NJ (US); Hien Nguyen, Est Windsor, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/179,497

(22) Filed: Jun. 25, 2002

(51) Int. Cl. A61F 13/15; A61F 13/20

(52) U.S. Cl. 604/374

(57) ABSTRACT

A novel absorbent structure for use in disposable absorbent articles includes a nonwoven fibrous web comprising at least about 5 wt-% of cellulosic materials, having a local density of greater than about 0.2 g/cm³, and a density relaxation of less than about 20%.

A novel process for forming a densified nonwoven web includes forming an open structure comprising at least about 5 wt-% of cellulosic materials; heating at least a portion of the open structure to a temperature of at least about 40° C.; compressing the heated open structure to form the densified nonwoven web to a local density of greater than about 0.2 g/cm³; and releasing the densified nonwoven web from compression.

### CROSS-REFERENCE TO RELATED APPLICATIONS

This invention is related to the following copending application: U.S. Ser. No. ______, filed Jun. 25, 2002, entitled “Compressed Absorbent Tampon” (Att'y Docket, PPC-841).

### FIELD OF THE INVENTION

The present invention relates to a process and apparatus for forming a densified nonwoven web using relatively low compression forces. The process includes heating an open structure prior to compression, and the apparatus includes elements for this heating.

### BACKGROUND OF THE INVENTION

Absorbent structures are manufactured under compression to provide sufficient absorbent capacity for a given use in a conveniently dimensioned product. Absorbent structures may include wound care, diapers, sanitary napkins, tampons, pantiliners, interlabial devices, incontinence articles, meat tray liners, shoe liners, and the like.

Many absorbent structures, such as tampons and absorbent webs, achieve shape stability by slightly overcompressing the structure and allowing it to recover or expand to the desired dimensions. This structure may also be heat set. An example of this is described in Johst et al., U.S. Pat. No. 4,081,884. This patent discloses radially compressing a tampon blank comprising cellulosic fibers, introducing the radially compressed tampon blank into a heated chamber, and axially compressing the tampon while heating for at least about five seconds. This process requires significant time to set the tampon.

Manufacturing methods for absorbent webs often include a press to densify the webs to a desired degree. An example of this is shown in Tan et al., U.S. Pat. No. 5,916,670, that employs a heated calender to compress the material to form a web having a desired density.

While the force required for compression required in these references is not discussed in any great detail, it is our experience that the energy required to radially compress a fibrous web, as measured by the compressive force, is very great. This compressive force can also limit the ability to commercially produce fibrous webs having increased density without damaging process equipment or the fibrous structure and consequently the absorbent capacity of the web due to the excessive forces involved in compressing the web.

Therefore, what is needed is a densified absorbent fibrous web with low density relaxation (as defined in the Examples). What is also needed is a process that can provide such a densified web using lower compressive force to reduce the risk of web and equipment damage.

### SUMMARY OF THE INVENTION

It is an object of the present invention to provide a densified absorbent fibrous web with low density relaxation. This permits production of higher density structures or parts of structures, without significant material damage.

It is another object of the present invention to provide process that can provide a densified absorbent fibrous web with low density relaxation using lower compressive force than would otherwise be required to reduce the risk of web and equipment damage.

In accordance with the present invention, there has been provided a novel absorbent structure for use in disposable absorbent articles. The structure includes a nonwoven fibrous web comprising at least about 5 wt-% of cellulosic materials, having a local density of greater than about 0.2 g/cm³, and a density relaxation of less than about 20%.

The present invention also relates to a novel process for forming a densified nonwoven web. The process includes forming an open structure comprising at least about 5 wt-% of cellulosic materials; heating at least a portion of the open structure to a temperature of at least about 40° C.; compressing the heated open structure to form the densified nonwoven web to a local density of greater than about 0.2 g/cm³; and releasing the densified nonwoven web from compression.

### BRIEF DESCRIPTION OF THE DRAWING

FIG. 1 is a top plan view of a sanitary napkin incorporating a densified absorbent web according to the present invention.

FIG. 2 is a diagrammatic view of a conventional apparatus and process for producing a densified absorbent web.

FIG. 3 is a diagrammatic view of an apparatus and process for producing a densified absorbent web according to an embodiment of the present invention.

FIG. 4 is a diagrammatic view of an apparatus and process for producing a densified absorbent web according to another embodiment of the present invention.

FIG. 5 is a diagrammatic view of an apparatus and process for producing a densified absorbent web according to yet another embodiment of the present invention.

FIG. 6 is a diagrammatic view of an apparatus and process for producing a densified absorbent web according to an embodiment of the present invention, combining features of the embodiments of FIGS. 4 and 5.

### DETAILED DESCRIPTION OF THE PREFERRED EMBODIMENTS

Further characteristics and advantages of the invention will become clear from the following detailed description, appended drawings, and non-limiting examples.

As used in the specification and claims, the term “open structure” and variations of this term relate to compressible structures prior to substantial compression to form a densified web. For example, these open structures may be formed by carding, air laying, or other processes and may include some minor calendering to maintain a density of less than about 0.1 g/cm³.

As used in the specification and claims, the term “compressible” and variations of this term relates to structures that can be compressed to hold a generally compressed form and that also can expand to a relatively uncompressed state upon exposure to sufficient moisture or liquid.

The present invention is directed to novel absorbent structures, useful in disposable absorbent articles such as, for example, sanitary napkins, pantiliners, diapers, incontinence articles, wound care bandages, and the like. The absorbent structures have a nonwoven fibrous web comprising at least about 5 wt-% of cellulosic materials, having a local density of greater than about 0.2 g/cm³, and a density relaxation of less than about 20%. The absorbent fibrous web may optionally include non-cellulosice materials, such as fibers, superabsorbent materials, and the like. The fibrous web preferably has a high density and in a specific example has a local density of greater than about 0.2 g/cm³. Preferably, the second absorbent layer 48 may have a density in the range of from about 0.25 g/cm³ to about 0.4 g/cm³. More preferably, the density is from about 0.25 g/cm³ to about 0.35 g/cm³.

The absorbent fibrous web may have a substantially uniform density as a result of compression by, e.g., smooth calender rolls, or it may have at least one portion of increased density as a result of an embossing process. If the web is embossed, higher levels of bulk may be maintained. Such structures are useful for some diapers and other bulkier absorbent products. Of course, the local densified portions benefit from the lower forces necessary to compress the portions and improved density relaxation of less than 20%.

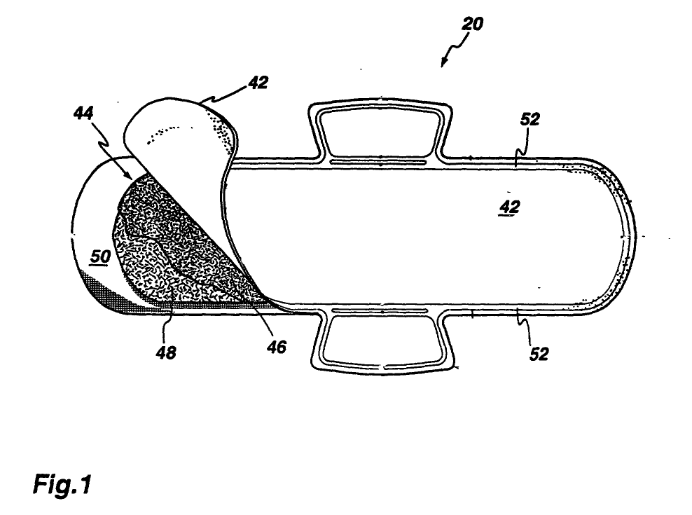

This absorbent structure can be used in disposable absorbent articles. Examples of such disposable absorbent articles includes, without limitation, sanitary napkins, diapers, incontinence articles, bandages and other wound care articles, tampons, pantiliners, shoe inserts, meat tray liners, and the like. These articles are adapted to be placed in direct contact with the body for the purpose of absorbing body fluids and are subsequently thrown away after a single use. These disposable absorbent articles generally are formed of at least a cover layer, an absorbent system and a barrier layer. The absorbent system can be formed of an individual layer or it can be formed of two or more layers, a first absorbent layer and a second absorbent layer. FIG. 1 illustrates an exemplary sanitary napkin 20 formed in part of a densified absorbent web of the present invention. The sanitary napkin 20 includes a cover layer 42, an absorbent system 44, and a barrier layer 50.

The cover layer 42 may be a relatively low density, bulky, high-loft non-woven web material. The cover layer 42 may be composed of only one type of fiber, such as polyester or polypropylene or it may be composed of bi-component or conjugate fibers having a low melting point component and a high melting point component. The fibers may be selected from a variety of natural and synthetic materials such as nylon, polyester, rayon (in combination with other fibers), cotton, acrylic fiber and the like and combinations thereof. An example is the non-woven cover layer of sanitary napkins sold by Johnson & Johnson Inc. of Montreal, Canada under the trademark Stayfree Ultra-Thin Cottony Dry Cover.

Bi-component fibers may be made up of a polyester core and a polyethylene sheath. The use of appropriate bi-component materials results in a fusible non-woven fabric. Examples of such fusible fabrics are described in U.S. Pat. No. 4,555,446 issued Nov. 5, 1985 to Mays. Using a fusible fabric increases the ease with which the cover layer may be mounted to the adjacent first absorbent layer and/or to the barrier layer.

The cover layer 42 preferably has a relatively high degree of wettability, although the individual fibers comprising the cover may not be particularly hydrophilic. The cover material should also contain a great number of relatively large pores. This is because the cover layer 42 is intended to take-up body fluid rapidly and transport it away from the body and the point of deposition. Advantageously, the fibers which make up the cover layer 42 should not lose their physical properties when they are wetted, in other words they should not collapse or lose their resiliency when subjected to water or body fluid. The cover layer 42 may be treated to allow fluid to pass through it readily. The cover layer 42 also functions to transfer the fluid quickly to the other layers of the absorbent system 44. Thus, the cover layer 42 is advantageously wettable, hydrophilic and porous. When composed of synthetic hydrophobic fibers such as polypropylene or bi-component fibers, the cover layer 42 may be treated with a surfactant to impart the desired degree of wettability.

Alternatively, the cover layer 42 can also be made of polymer film having large pores. Because of such high porosity, the film accomplishes the function of quickly transferring body fluid to the inner layers of the absorbent system. Apertured co-extruded films such as described in U.S. Pat. No. 4,690,679 and available on sanitary napkins sold by Johnson & Johnson Inc. of Montreal, Canada could be useful as cover layers in the present invention.

The cover layer 42 may be embossed to the remainder of the absorbent system 44 in order to aid in promoting fluid transport by fusing the cover to the next layer. Such fusion may be effected locally, at a plurality of sites or over the entire contact surface of cover layer 42 with absorbent system 44. Alternatively, the cover layer 42 may be attached to the absorbent system 44 by other means such as by adhesive.

Adjacent to the cover layer 42 on its inner side and preferably bonded to the cover layer 42 is a first absorbent layer 46 that forms part of the absorbent system 44. The first absorbent layer 46 provides the means of receiving body fluid from the cover layer 42 and holding it until an underlying second absorbent layer has an opportunity to absorb the fluid.

The first absorbent layer 46 may be composed of fibrous materials, such as wood pulp, polyester, rayon, flexible foam, or the like, or combinations thereof. The first absorbent layer 46 may also comprise thermoplastic fibers for the purpose of stabilizing the layer and maintaining its structural integrity. The first absorbent layer 46 may be treated with surfactant on one or both sides in order to increase its wettability, although generally the first absorbent layer 46 is relatively hydrophilic and may not require treatment. The first absorbent layer 46 is preferably bonded on both sides to the adjacent layers, i.e. the cover layer 42 and an underlying second absorbent layer 48. An example of a suitable first absorbent layer is a through air bonded pulp sold by BUCKEYE of Memphis Tenn. under the designation VIZORB 3008.

Immediately adjacent to and preferably bonded to the first absorbent layer 46 is the second absorbent layer 48. The second absorbent layer 48 is formed of the densified absorbent web of the present invention. Its composition and construction is described in greater detail hereafter.

Underlying the absorbent system 44 is a barrier layer 50 comprising liquid-impervious film material so as to prevent liquid that is entrapped in the absorbent system 44 from egressing the sanitary napkin and staining the wearer's undergarment. The barrier layer 50 is made preferably of polymeric film.

The cover layer 42 and the barrier layer 50 are joined along their marginal portions so as to form an enclosure or flange seal that maintains the absorbent system 44 captive. The joint may be made by means of adhesives, heat-bonding, ultrasonic bonding, radio frequency sealing, mechanical crimping, and the like and combinations thereof. The peripheral seal line is shown in FIG. 1 by the reference numeral 52.

Absorbent Web

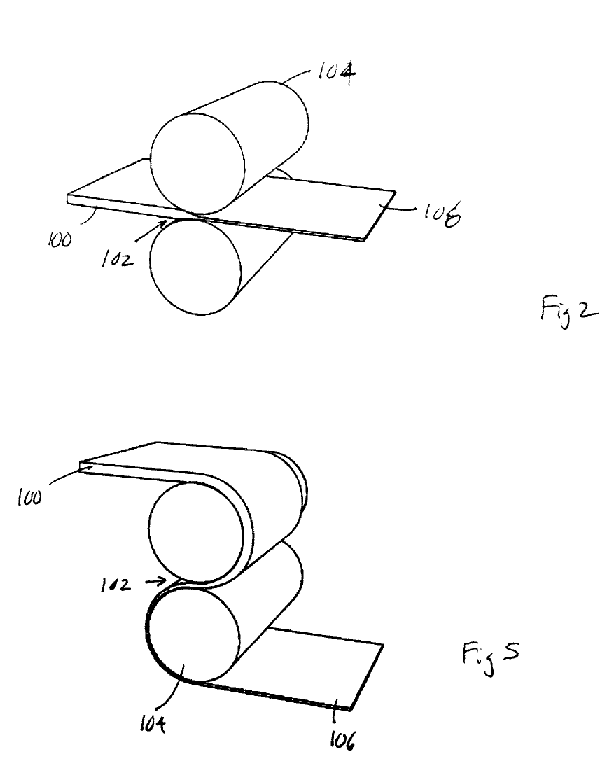

Densified absorbent webs are generally formed by compressing an open structure to form a thinner product having a higher density than the open structure. After the web is released from compression, it expands, slightly, to its final dimensions. The densified web may have a generally uniform density, or it may have regions of differing density. An illustration of such a conventional process is shown in FIG. 2 in which an open structure 100 is passed through the nip 102 of a pair of calendar rolls 104 to form a densified absorbent web 105. The calendar rolls 104 can be heated, cooled, or maintained at substantially room temperature.

The densified webs of the present invention can be formed in processes shown in FIGS. 3-6, and these processes are discussed in greater detail hereafter. The open structure 100 that will form the densified absorbent web of the present invention 106 is a material containing at least about 5 wt-% cellulosic materials. These materials are moisture sensitive, and provide hydrogen bonding when compressed under moist conditions. More preferably, the densified absorbent web includes about 35 to about 100 wt-% cellulosic materials, and most preferably, about 50 to about 75 wt-% cellulosic materials. The densified absorbent webs can also include other non-cellulosic materials including, without limitation, polyesters, polyvinyl alcohols, polyolefins, polyamines, polyamides, polyacrylonitriles, SAP (superabsorbent polymers), hydrogels, and the like. These non-cellulosic materials can be present at up to about 95 wt-% of the densified absorbent web. More preferably, the non-cellulosic materials are present at about 0 to about 65 wt-%, and most preferably, about 25 to about 50 wt-% of the densified absorbent web. As used herein, the phrase “wt-%” means weight of substance per weight of final material. By way of example, 10 wt-% SAP means 10 g/m2 SAP per 100 g/m2 basis weight of the material.

Prior to heating, the open structure 100 has a moisture content of at least about 4 wt-%, preferably, about 8 to about 13 wt-%. After heating, the open structure retains sufficient moisture content to promote interfiber bonds sufficient to maintain the dimensions of the densified absorbent web. Preferably, the densified absorbent web has a moisture content of less than about 13 wt-%, more preferably, less than about 10 wt-%, and most preferably, from about 2 to about 10 wt-%.

The materials that may be used in the web include fibers, foams, and particles or other discrete materials. Cellulosic materials that can be used in the open structure 100 are well known in the art and include natural fibers such as wood pulp, cotton, flax, jute, hemp, peat moss, and the like. Cellulosic materials can also include processed materials including cellulose derivatives such as regenerated cellulose (including viscose rayon and lyocel), cellulose nitrate, carboxymethyl cellulose, and the like. Wood pulp can be obtained from mechanical or chemi-mechanical, sulfite, kraft, pulping reject materials, organic solvent pulps, etc. Both softwood and hardwood species are useful. Softwood pulps are preferred. It is not necessary to treat cellulosic fibers with chemical debonding agents, cross-linking agents and the like for use in the present material.

Preferably, the webs have a significant proportion of fibers. The fibers may be any of the materials listed above, and may have any useful cross-section, including multi-limbed and non-limbed. Multi-limbed, regenerated cellulosic fibers have been commercially available for a number of years. These fibers are known to possess increased specific absorbency over non-limbed fibers. Commercial examples of these fibers are Danufil VY trilobal viscose rayon fibers available from Acordis Ltd., Spondon, England. These fibers are described in detail in Wilkes et al, U.S. Pat. No. 5,458,835, the disclosure of which is hereby incorporated by reference.

The open structure 100 can contain any superabsorbent polymer (SAP), which SAPs are well known in the art. For the purposes of the present invention, the term “superabsorbent polymer” (or “SAP”) refers to materials which are capable of absorbing and retaining at least about 10 times their weight in body fluids under a 0.5 psi pressure. The superabsorbent polymer particles of the invention may be inorganic or organic crosslinked hydrophilic polymers, such as polyvinyl alcohols, polyethylene oxides, crosslinked starches, guar gum, xanthan gum, and the like. The particles may be in the form of a powder, grains, granules, or fibers. Preferred superabsorbent polymer particles for use in the present invention are crosslinked polyacrylates, such as the product offered by Sumitomo Seika Chemicals Co., Ltd. Of Osaka, Japan, under the designation of SA70*.

The densified absorbent web 106 can be prepared over a wide range of basis weights. It can have a basis weight in the range of from about 40 g/m² about 700 g/m². In a specific example, the basis weight ranges from about 150 g/m² to about 350 g/m². Preferably the basis weight ranges from about 200 g/m² to about 300 g/m² and, more preferably, to about 250 g/m².

The densified absorbent web 106 can be formed as three or four lamina or strata. Those strata include a bottom layer, one or two middle layers and a top layer. Specific examples of three and four layer material are set forth below. The SAP can be included in any or all of the layers. The concentration (wt-%) of SAP in each layer can vary as can the nature of the particular SAP.

Even where prepared as from multiple layers, the final thickness of the formed densified absorbent web 106 is low. The thickness of the densified or embossed portions can vary from about 0.5 mm to about 2.5 mm. In a specific example, the thickness is from about 1.0 mm to about 2.0 mm and, even more specifically from about 1.25 mm to about 1.75 mm. Of course, if only portions of the web are densified or embossed, un-densified areas may have a substantially greater thickness. Thus, an embossed web may have an overall thickness of greater than about 5 mm.

Process The process of the present invention begins with an open structure. The open structure may be a nonwoven web, a mass of randomly or substantially uniformly oriented materials, such as fibers, foams, or particles, and the like.

A nonwoven web useful in the present invention can be formed in any manner desired by the person of ordinary skill in the art. For example, fibers can be opened and/or blended by continuously metering them into a saw-tooth opener. The blended fibers can be transported, e.g., by air through a conduit to a carding station to form a fibrous web. Alternatively, a mass of substantially randomly oriented fibers can be formed by opening and/or blending them, transporting them, as above, to a station to form, e.g., an air-laid open structure.

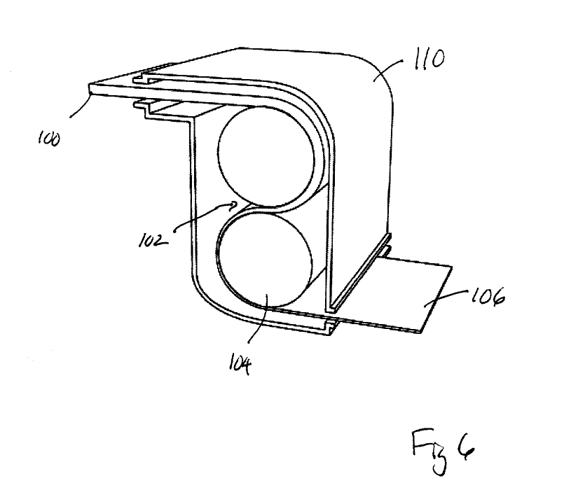

Air-laid absorbents are typically produced with a low density. To achieve higher density levels, such as the examples of the second absorbent layer 48 given above, the air-laid material is compacted using calenders as shown in FIG. 5. Compaction is accomplished using means well known in the art. Typically such compaction is carried out at a temperature of about 100 degrees C. and a load of about 130 Newtons per millimeter. The upper compaction roll is typically made of steel while the lower compaction roll is a flexroll having a hardness of about 85 SH D. It is preferred that both the upper and lower compaction rolls be smooth, although the upper roll can be engraved.

FIG. 3 shows the addition of heaters 108 proximate the open structure 100. The heaters 108 can apply heat to the open structure 100 via circulation of hot air or steam, electromagnetic transmission of energy (for example, without limitation, radio frequency energy, infrared energy, ultrasonic energy, microwave energy, and the like), vibration (for example, ultrasonic energy and the like), insertion of heated pins into web to provide conductive heat transfer, and the like. It may also be possible to apply radiant heat, although it is somewhat less effective and may require dwell times in excess of one second.

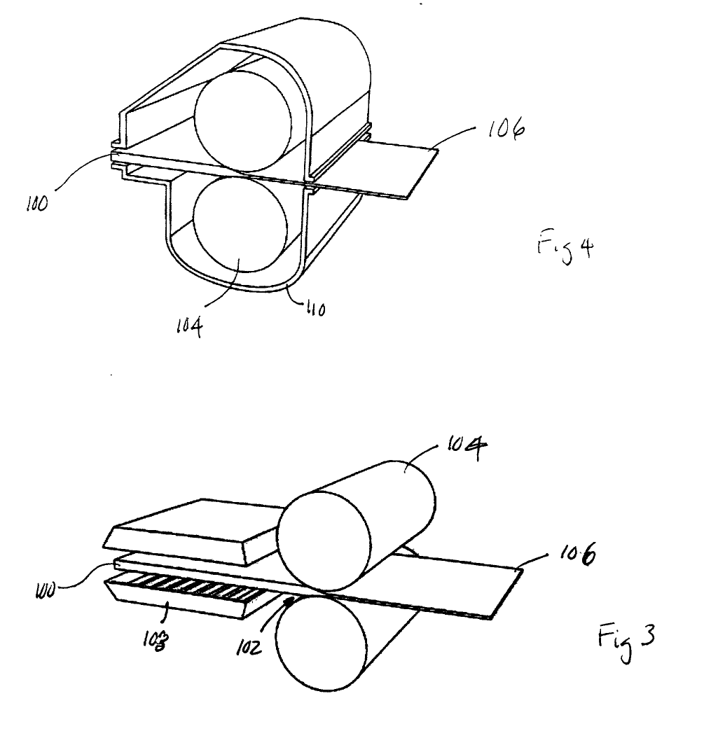

FIG. 4 shows an alternative process in which the open structure 100, heated calendar rolls 104, and densified web 106 are enclosed in an enclosure 110 which traps heat from the calendar rolls 104 and moisture from the web. The heat trapped within the enclosure 110 can then preheat the incoming open structure 100. Of course, this can be enhanced by additional heating means (not shown) which act either upstream of or within the enclosure 110.

FIG. 5 shows yet another alternative process in which the web passes around the calendar rolls 104 in an S-shaped path. In this manner, the open structure 100 is pre-heated by the first calendar roll 104a before passing through the nip 102 of the first and second calendar rolls 104a, 104b to form the densified absorbent web 106.

Finally, FIG. 6 shows a combination of the process of FIGS. 4 and 5. In this embodiment, the web travels the S-shaped path around the heated calendar rolls, and the apparatus is enclosed by the enclosure 110 to retain the heat and moisture to further improve the pre-heating of the open structure 100. It is believed that the retained heat and moisture improve the ability of the cellulosic material in the web to form hydrogen bonds.

Heat can be applied to the fibrous web via conduction, convection, radiation, and the like. Such processed include, without limitation, circulation of hot air or steam, electromagnetic transmission of energy (for example, without limitation, radio frequency energy, infrared energy, microwave energy, and the like), insertion of heated pins into web to provide conductive heat transfer, ultrasonic energy, and the like. In a preferred process, the heat is applied via circulation of hot air and/or providing an enclosure for heated calender rolls and the upstream web. The open structure is heated to a temperature of at least about 40° C. More preferably, the open structure is heated to at least about 45° C., and most preferably, the open structure is heated to at least about 60° C. To achieve a temperature of about 40° C. to about 45° C., calender rolls can be maintained at about 100° C., while maintaining the calender rolls at about 140° C. can provide an open structure temperature of about 100° C. In order to avoid over-heating some thermoplastic fibers or over-drying the structure, it may also be beneficial to limit the temperature of the open structure to less than about 100° C. or even 85° C.

Surprisingly, both the force required for compression and degree of over-compression are significantly reduced when the fibrous web is heated prior to compression. Indeed, what we have found is that heating the fibrous web prior to compression into the densified absorbent web provides a more consistent, dimensionally-controlled product. It also requires lower compression forces to achieve a dimensionally stable product.

One way to illustrate the consistency and dimensional control of the densified web is a review of the density relaxation (as defined below in the Examples) of the web. Preferably, the webs of the present invention have a density relaxation of less than about 20%, more preferably, less than about 10%, and most preferably, less than about 5%.

Another way to illustrate the advantages of the present invention is the reduced fiber damage in the web. Fiber damage, including permanent fiber deformation and breakage, occurs during compression of the web. Fiber damage can be determined by examining the tampon for fibers that have been broken. For example, webs that are formed of staple length fibers (about 1 to 1.5 inches (25 to about 40 mm)) can be inspected to determine the number or percentage of fibers that have a length of less than about ¾ inch (18 mm). Alternatively, these webs can be analyzed to determine the percentage of fines (fibers having a length of less than about ¼ inch (7 mm)). A significant percentage of short fibers or fines can be indicative of fiber damage in a product.

After heating, the open structure 100 beneficially retains its heat due to the inherent insulating properties of a loosely gathered mass of fibers and the heated air trapped in the capillaries thereof. Looser capillaries of the more open web allow more even heat transfer into center of the web.

We also believe that the present process allows for increased manufacturing line speeds and improved processability. For example, we have found that early fiber heating decreases the amount of materials, such as fibers, lost from the open structure during pre-compression handling. This produces a more consistent material stream leading into later processing stations.

### EXAMPLES

The present invention will be further understood by reference to the following specific Examples that are illustrative of the composition, form and method of producing the device of the present invention. It is to be understood that many variations of composition, form and method of producing the device would be apparent to those skilled in the art. The following Examples, wherein parts and percentages are by weight unless otherwise indicated, are only illustrative.

### Example 1

A blend of 75 wt-% 3 denier Danufil® VY trilobal viscose rayon fibers and 25 wt-% 3 denier Danufil® V viscose rayon fibers, both available from Acordis Ltd., Spondon, England, were opened via standard fiber opening and carding equipment. A fixed amount of the fiberblend (having a mass, W, of about 2 g) was introduced in a stainless steel mold with a cylindrical cavity (of cross-sectional area, A, of about 5 cm²). A cylindrical plunger size matched to the cylindrical cavity was used to compress the fiber mass using a standard laboratory press. In order to heat the samples, the mold and plunger were heated together in an oven set at the target temperature. After sufficient time to allow the mold and plunger to reach the oven temperature, the fibers were placed into the cavity, and the mold, plunger and fibers were heated for an additional three minutes to allow the fibers to reach the oven temperature. The heated assembly was removed from the oven and placed between the plates of the laboratory press. Pressure was applied to compress the fiber mass in the cavity up to a predetermined peak pressure and released, after which the compressed fibrous plug was removed to allow an immediate measurement of the initial thickness, T₀.

The compressed fibrous plug had an initial volume (V₀=A*T₀) and an initial density (ρ₀=W/V₀) but the plug expanded after the pressure was released, reaching equilibrium after about 15 to 20 minutes (at room temperature, approximately 20° C.). While the humidity conditions of the test are not generally critical, conducting the test at high humidity will adversely affect the test results. The equilibrium thickness, Tₑ, was then measured to provide an equilibrium density (ρₑ=W/(A*Tₑ)). From these values, a density relaxation can be calculated equal to (ρ₀−ρₑ)/ρ₀.

A control was prepared employing mold, plunger and fibers at room temperature, about 20° C. The results of measurements at each temperature and pressure are shown in Table 1.

TABLE 1

| | | | Equilibrium | % Density |
|---|---|---|---|---|
| Temperature | Peak Pressure | Initial Density | Density | relaxation |
| 100° C. | 610 | 0.46 | 0.45 | 2% |
| | 1200 | 0.62 | 0.62 | <2% |
| | 1800 | 0.75 | 0.75 | <2% |
| | 2500 | 0.80 | 0.80 | <2% |
| | 3000 | 0.85 | 0.84 | <2% |
| | 3600 | 0.88 | 0.88 | <2% |
| | 4800 | 0.92 | 0.92 | <2% |
| | 6100 | 0.95 | 0.95 | <2% |
| 85° C. | 610 | 0.34 | 0.34 | <2% |
| | 910 | 0.49 | 0.49 | <2% |
| | 1200 | 0.43 | 0.43 | <2% |
| | 1500 | 0.55 | 0.55 | <2% |
| | 1800 | 0.53 | 0.53 | <2% |
| | 3600 | 0.85 | 0.86 | <2% |
| | 4900 | 0.85 | 0.86 | <2% |
| 75° C. | 610 | 0.36 | 0.36 | <2% |
| | 910 | 0.40 | 0.39 | 3% |
| | 1200 | 0.52 | 0.51 | 2% |
| | 1500 | 0.54 | 0.53 | <2% |
| | 1800 | 0.65 | 0.64 | <2% |
| | 3600 | 0.80 | 0.80 | <2% |
| | 4900 | 0.84 | 0.84 | <2% |
| | 6100 | 0.91 | 0.92 | <2% |
| 60° C. | 910 | 0.33 | 0.32 | 3% |
| | 1200 | 0.41 | 0.40 | 2% |
| | 1500 | 0.44 | 0.44 | <2% |
| | 1800 | 0.49 | 0.47 | 4% |
| | 2200 | 0.57 | 0.55 | 4% |
| | 3000 | 0.65 | 0.64 | <2% |
| | 4900 | 0.79 | 0.78 | <2% |
| | 6100 | 0.87 | 0.87 | <2% |
| 40° C. | 1200 | 0.31 | 0.30 | 3% |
| | 1800 | 0.43 | 0.40 | 7% |
| | 2400 | 0.50 | 0.47 | 6% |
| | 3000 | 0.58 | 0.56 | 3% |
| | 4300 | 0.69 | 0.66 | 4% |
| ROOM | 1200 | 0.23 | 0.17 | 26% |
| | 2400 | 0.33 | 0.25 | 24% |
| | 3600 | 0.50 | 0.38 | 24% |
| | 4900 | 0.60 | 0.46 | 23% |
| | 6200 | 0.64 | 0.51 | 20% |

These data show that pre-heating of fibers at a temperature of at least about 40° C. and maintaining the heat during compression provides a significantly greater dimensional stability than compressing the same fibers at room temperature. They further illustrate that substantially higher fiber plug densities can be achieved at lower compression pressures when fibers are pre-heated. This is even more pronounced at temperatures of greater than about 60° C.

### Example 2

The procedure of Example 1 was repeated with a blend of 75 wt-% 3 denier Danufil® V viscose rayon fibers, available from Acordis Ltd. (Spondon, England), and 25 wt-% 3 denier T-224 polyester fibers, available from KoSa, (Houston, Tex., USA). Again, the results of the measurements at each temperature and pressure are shown in Table 2.

TABLE 2

| | | | Equilibrium | % Density |
|---|---|---|---|---|
| Temperature | Peak Pressure | Initial Density | Density | relaxation |
| 100° C. | 610 | 0.33 | 0.34 | <2% |
| | 1200 | 0.47 | 0.48 | <2% |
| | 7400 | 0.62 | 0.63 | <2% |
| | 3000 | 0.65 | 0.65 | <2% |
| 85° C. | 910 | 0.36 | 0.35 | 3% |
| | 1200 | 0.39 | 0.39 | <2% |
| | 2400 | 0.53 | 0.53 | <2% |
| | 3600 | 0.66 | 0.66 | <2% |
| | 4900 | 0.80 | 0.79 | <2% |
| | 6100 | 0.79 | 0.78 | <2% |
| 75° C. | 910 | 0.33 | 0.32 | 3% |
| | 1200 | 0.37 | 0.37 | <2% |
| | 2400 | 0.52 | 0.50 | 4% |
| | 3600 | 0.65 | 0.64 | <2% |
| | 4900 | 0.71 | 0.70 | <2% |
| | 6100 | 0.79 | 0.80 | <2% |
| 60° C. | 610 | 0.25 | 0.24 | 4% |
| | 1200 | 0.38 | 0.37 | 3% |
| | 2400 | 0.50 | 0.50 | <2% |
| | 3600 | 0.62 | 0.60 | 3% |
| | 6100 | 0.77 | 0.75 | 3% |
| 45° C. | 910 | 0.30 | 0.29 | 3% |
| | 1200 | 0.34 | 0.34 | <2% |
| | 2400 | 0.44 | 0.43 | 2% |
| | 3600 | 0.59 | 0.59 | <2% |
| | 4900 | 0.71 | 0.69 | 3% |
| | 6100 | 0.77 | 0.76 | <2% |
| ROOM | 2400 | 0.41 | 0.33 | 20% |
| | 3600 | 0.51 | 0.40 | 22% |
| | 3800 | 0.55 | 0.40 | 27% |
| | 3800 | 0.52 | 0.40 | 23% |
| | 4900 | 0.61 | 0.52 | 15% |
| These data show that pre-heating of fibers at a temperature of at least about 45° C. and maintaining the heat during compression provides a significantly greater dimensional stability than compressing the same fibers at room temperature. They further illustrate that substantially higher fiber plug densities can be achieved at lower compression pressures when fibers are pre-heated. This is even more pronounced at | | | | |
| # temperatures of greater than about 60° C. However, with thermoplastic fibers, such as polyester fibers, this preheating may be limited to avoid exceeding their yield point to cause permanent deformation of the fibers, including melting the fibers. | | | | |

### Example 3

The procedure of Example 1 was repeated with different blends of 3 denier Danufil® V viscose rayon fibers, available from Acordis Ltd. (Spondon, England), and 3 denier T-224 polyester fibers, available from KoSa, (Houston, Tex., USA). However, in this series, the temperature was maintained at 75° C., while the proportion of fibers varied. The results of the measurements at each blend and pressure are shown in Table 3.

TABLE 3

| | | | Equilibrium | % Density |
|---|---|---|---|---|
| | Peak Pressure | Initial Density | Density | relaxation |
| 25% PET | 910 | 0.33 | 0.32 | 3% |
| | 1200 | 0.37 | 0.37 | <2% |
| | 2400 | 0.52 | 0.50 | 4% |
| | 3600 | 0.65 | 0.64 | <2% |
| | 4900 | 0.71 | 0.70 | <2% |
| | 6100 | 0.79 | 0.80 | <2% |
| 33% PET | 610 | 0.24 | 0.23 | 4% |
| | 910 | 0.31 | 0.30 | 3% |
| | 1200 | 0.39 | 0.38 | 3% |
| | 2400 | 0.49 | 0.47 | 4% |
| 50% PET | 910 | 0.28 | 0.28 | <2% |
| | 1200 | 0.32 | 0.31 | 3% |
| | 2400 | 0.49 | 0.47 | 4% |
| | 3600 | 0.59 | 0.57 | 3% |
| | 4900 | 0.68 | 0.67 | <2% |
| | 6100 | 0.75 | 0.75 | <2% |
| 67% PET | 1200 | 0.31 | 0.30 | 3% |
| | 2400 | 0.41 | 0.41 | <2% |
| | 3600 | 0.63 | 0.62 | <2% |
| | 6100 | 0.70 | 0.69 | <2% |

These data show that pre-heating of fibers at a temperature of about 75° C. and maintaining the heat during compression provides significant dimensional stability, even with large proportions of relatively resilient fibers, such as polyester.

The specification and embodiments above are presented to aid in the complete and non-limiting understanding of the invention disclosed herein. Since many variations and embodiments of the invention can be made without departing from its spirit and scope, the invention resides in the claims hereinafter appended.

What is claimed is:

1. A process for forming a densified nonwoven web comprising the steps of:
forming an open structure comprising at least about 5 wt-% of cellulosic materials;
heating the open structure to a temperature of at least about 40° C. while substantially maintaining moisture present in the open structure;
compressing at least a portion of the heated open structure to form the densified nonwoven web having portions with a local density of greater than about 0.2 g/cm³; and
releasing the densified nonwoven web from compression.

2. The process of claim 1 wherein the open structure comprises at least about 15 wt-% of cellulosic materials.

3. The process of claim 1 wherein the open structure further comprises at least about 5 wt-% non-cellulosic polymeric materials.

4. The process of claim 1 comprising heating the open structure to a temperature of at least about 45° C.

5. The process of claim 4 comprising heating the open structure to a temperature of at least about 60° C.

6. The process of claim 1 wherein the step of heating the open structure comprises maintaining at least about 2 wt-% moisture in the open structure.

7. The process of claim 1 wherein the step of compressing the heated open structure forms portions having a local density of about 0.25 to about 0.35 g/cm³.

8. The process of claim 1 wherein the cellulosic materials comprise cellulosic fibers.

9. The process of claim 3 wherein the non-cellulosic polymeric materials comprise fibers.

10. The process of claim 3 wherein the non-cellulose polymeric materials comprise superabsorbent materials.

11. The process of claim 10 wherein the superabsorbent materials are particulate.

12. The process of claim 10 wherein the superabsorbent materials are fibers.

13. The process of claim 1 wherein the step of heating the open structure comprises moving heated air through the open structure.

14. The process of claim 13 wherein the heated air is humidified.

15. The process of claim 1 wherein the step of heating the open structure comprises transmitting electromagnetic energy to the open structure.

16. The process of claim 15 wherein the electromagnetic energy is selected from the group consisting of radio frequency energy, infrared energy, ultrasonic energy, and microwave energy.

17. The process of claim 1 further comprising the step of placing the densified nonwoven web between a liquid-pervious cover and a liquid impervious barrier.

18. The process of claim 1 wherein the at least a portion of the heated open structure comprises substantially all of the heated open structure, and the densified nonwoven web has a substantially uniform density of greater than about 0.2 g/cm³.

19. The process of claim 1 wherein the at least a portion of the heated open structure comprises one or more discrete portions that form embossed portions of the densified nonwoven web.

20. The process of claim 19 wherein the emobossed portions of the densified nonwoven web are lines.

21. An absorbent structure comprising a densified nonwoven fibrous web comprising at least about 5 wt-% of cellulosic materials, having a local density of greater than about 0.2 g/cm³, and a density relaxation of less than about 20% in densified portions.

22. The absorbent structure of claim 21 wherein the nonwoven fibrous web comprises at least about 15 wt-% of cellulosic materials.

23. The absorbent structure of claim 21 wherein the cellulosic materials comprise cellulosic fibers.

24. The absorbent structure of claim 21 wherein the nonwoven fibrous web further comprises at least about 5 wt-% non-cellulosic polymeric materials.

25. The absorbent structure of claim 24 wherein the non-cellulosic polymeric materials comprise fibers.

26. The absorbent structure of claim 24 wherein the non-cellulosic polymeric materials comprise superabsorbent materials.

27. The absorbent structure of claim 26 wherein the superabsorbent materials are particulate.

28. The absorbent structure of claim 21 wherein the nonwoven fibrous web has a moisture content of at least about 2 wt-%.

29. The absorbent structure of claim 21 wherein the nonwoven fibrous web has a density of greater than about 0.4 g/cm³.

30. The absorbent structure of claim 21 wherein the nonwoven fibrous web is enclosed between a liquid-pervious cover and a liquid impervious barrier.

31. Apparatus for forming a densified absorbent web comprising:
an open structure support
an open structure heater
a press that is suitable to compress the open structure into a densified absorbent web and that has an open structure infeed and a densified absorbent web outfeed
wherein the open structure support is associated with the open structure infeed and the open structure heater is associated with the open structure support whereby the open structure can be heated to a temperature significantly above ambient temperatures prior to introduction into the press.

32. Apparatus of claim 31 wherein the open structure support is arranged and configured to support a continuous fibrous web.

33. Apparatus of claim 32 wherein the open structure support is substantially planar.

34. Apparatus of claim 31 wherein the press is the nip between a pair of rolls.

35. Apparatus of claim 34 wherein the rolls are smooth.

36. Apparatus of claim 34 wherein at least one roll is profiled.

37. Apparatus of claim 36 wherein the rolls are embossing rolls.

38. Apparatus of claim 34 wherein the open structure support is one of the pair of rolls.

39. Apparatus of claim 31 wherein the open structure heater comprises an enclosure for the open structure support.

40. Apparatus of claim 39 wherein the open structure heater further comprises a heating element associated with the press.

41. Apparatus of claim 40 wherein the open structure heater comprises a heated calendar roll contained within the enclosure for the open structure support.

42. Apparatus of claim 41 wherein the open structure support is the heated calendar roll.
